(19) United States
(12) Patent Application Publication
(10) Pub. No.: US 2026/0358317 A1
(43) Pub. Date: Nov. 12, 2026

(54) AIR QUALITY MODELLING AND ESTIMATION METHODS

(71) Applicant: British Telecommunications Public Limited Company, London Greater London (GB)

(72) Inventors: Ian THURLOW, London (GB); Ian NEILD, London (GB); Ryan SHIMMON, London (GB)

(21) Appl. No.: 19/139,244

(22) PCT Filed: Dec. 4, 2023
(86) PCT No.: PCT/EP2023/084165

§ 371 Date: Jun. 13, 2025

(30) Foreign Application Priority Data
Dec. 22, 2022 (EP) 22216016.0
Dec. 22, 2022 (GB) 2219540.8

(51) Int. Cl. G01N 33/00

(52) U.S. Cl. CPC G01N 33/0075; G01N 33/0031

(57) ABSTRACT

Some examples of air quality modelling and estimation methods performed by a remote server module are disclosed. The remote server module receives first air quality data from the field-based sensor, wherein the first air quality data was measured during a first time period and receives second air quality sensed by at least one mobile vehicle positioned at at least one location relative to the field-based sensor, wherein the second air quality data was measured during the first time period. The remote server module generates an air quality model for the geographical region using at least the first air quality data and the second air quality data. To provide an air quality estimation, the remote server module receives third air quality data from the field-based sensor, wherein the third air quality data was measured by the field-based sensor in a second time period, after the first time period and, using the model and the third air quality data to predict air quality in the geographical region.

The present disclosure relates to air quality measurement and estimation, including but not limited to air quality measurement and estimation in urban areas such as cities and towns.

### BACKGROUND

Air pollution is significant health hazard as it causes respiratory problems, lung diseases, and cardiovascular issues and can contribute to mental health issues and aggravate existing health conditions.

Accurate measurement and forecasting of air quality is therefore an important element of managing and mitigating the risks of air pollution. It also helps assess risks to the environment and the climate caused by poor air quality standards. Accurate forecasting can also lead to ease in planning day-to-day activities, avoiding locations with high alert areas, and implementing effective pollution control measures.

Positioning multiple fixed field-based air quality sensors in a geographical region of interest provides an indication of air pollution levels at discrete locations in the region and can be used to provide real-time air quality measurements in the vicinity close to the fixed field-based sensors.

The examples described herein are not limited to examples which solve problems mentioned in this background section.

### SUMMARY

Examples of preferred aspects and embodiments of the invention are as set out in the accompanying independent and dependent claims.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A first aspect of the disclosed technology is a method, performed by a remote server module, for estimating the air quality in a geographical region comprising a field-based sensor fixed in the region, the method comprising: receiving first air quality data from the field-based sensor, wherein the first air quality data was measured during a first time period; receiving second air quality data sensed by at least one mobile vehicle positioned at at least one location relative to the field-based sensor, wherein the second air quality data was measured during the first time period; generating an air quality model for the geographical region using at least the first air quality data and the second air quality data; receiving third air quality data from the field-based sensor, wherein the third air quality data was measured by the field-based sensor in a second time period, after the first time period; and using the model and the third air quality data to predict air quality in the geographical region. In this way air quality data over a geographical area is obtained in an accurate manner without having to use a high density of fixed field-based sensors. The approach is scalable to large urban areas where air quality is to be assessed.

In some preferred example embodiments, the method further comprising: receiving first climate data associated with the geographical region, wherein the climate data was measured during the first time period; and wherein generating the air quality model further comprises using the first climate data. By taking into account climate data improved accuracy is found.

In some preferred example embodiments, the method further comprising: wherein the third air quality data was measured at a time when air quality data from mobile vehicles in the geographical region was unavailable. Thus even where mobile vehicles are not available the process gives accurate air quality data.

In some preferred example embodiments, the method further comprising: receiving second climate data associated with the geographical region, wherein the second climate data was measured during the second time period; and using the second climate data, the model and the third air quality data to predict air quality in the geographical region.

In some preferred example embodiments, the method further comprising: wherein the first and/or second climate data is measured by the at least one mobile vehicle. Using a mobile vehicle to measure the climate data brings accuracy since the climate data is about the particular geographical region concerned.

In some preferred example embodiments, the method further comprising: wherein the mobile vehicle is an Unmanned Aerial Vehicle, UAV, and wherein the second air quality data is measured at a plurality of heights at the at least one location of the at least one UAV. Using air quality data from a plurality of heights gives improved accuracy since air quality may vary with height and UAVs are available a different heights.

In some preferred example embodiments, the method further comprising: calculating a height dependence of air quality in the geographical region using the second air quality data measured at the plurality of heights; wherein generating the air quality model further comprises using the height dependence of air quality.

In some preferred example embodiments, the method further comprising: wherein one or more lighter weight mobile vehicles of the at least one mobile vehicles and/or the field-based sensor are configured to send air quality data to a heavier weight mobile vehicle, wherein the first, second and third air quality data is received from the heavier weight mobile vehicle. The heavier weight mobile vehicles comprise at least one of improved communications hardware, larger data storage capacity and/or improved encryption capabilities, compared to the lighter weight mobile vehicles.

In some preferred example embodiments, the method further comprising: wherein the air quality model is a machine learning model. Using a machine learning model gives a particularly accurate outcome since there is a principled way to combine data of different types and sources.

Another aspect of the disclosed technology comprises a remote server module for estimating the air quality in a geographical region comprising a field-based sensor fixed in the region, the remote serve module configured to: receive first air quality data from the field-based sensor, wherein the first air quality data was measured during a first time period; receive second air quality data sensed by at least one mobile vehicle positioned at at least one location relative to the field-based sensor, wherein the second air quality data was measured during the first time period; generate an air quality model for the geographical region using at least the first air quality data and the second air quality data; receiving third air quality data from the field-based sensor, wherein the third air quality data was measured by the field-based sensor in a second time period, after the first time period; and using the model and the third air quality data to predict air quality in the geographical region.

In some preferred example embodiments, the remote server module further configured to: receive first climate data associated with the geographical region, wherein the climate data was measured during the first time period; and wherein generating the air quality model further comprises using the first climate data.

In some preferred example embodiments, the remote server module further configured to: receive the third air quality data from the field-based sensor, at a time when air quality data from mobile vehicles in the geographical region was unavailable.

In some preferred example embodiments, the remote server module further configured to: receive second climate data associated with the geographical region, wherein the second climate data was measured during the second time period; and using the second climate data, the model and the third air quality data to predict air quality in the geographical region.

In some preferred example embodiments, the remote server module further configured to: wherein the second air quality data was measured by a plurality of mobile vehicles in the geographical region and wherein the predicting air quality in the geographical region comprises predicting air quality at locations in the geographical region disparate from the field-based sensor and the mobile vehicles.

Another aspect of the disclosed technology comprises a computer implemented method for estimating air quality at a specified time and location in a geographical region comprising a field-based sensor fixed in the region, the method comprising: receiving air quality data from the field-based sensor, wherein the air quality data was measured by the field-based sensor at the specified time; inputting the air quality data and the location to a trained model to predict the air quality at the specified time and location; wherein the location is different from the location of the field-based sensor and wherein the model has been trained using air quality data measured by the field-based sensor and at least one mobile vehicle in the region.

It will also be apparent to anyone of ordinary skill in the art, that some of the preferred features indicated above as preferable in the context of one of the aspects of the disclosed technology indicated may replace one or more preferred features of other ones of the preferred aspects of the disclosed technology. Such apparent combinations are not explicitly listed above under each such possible additional aspect for the sake of conciseness.

Other examples will become apparent from the following detailed description, which, when taken in conjunction with the drawings, illustrate by way of example the principles of the disclosed technology.

### BRIEF DESCRIPTION OF THE DRAWINGS

FIG. 1 illustrates a plurality of UAVs positioned in a geographical region surrounding a field-based sensor;

FIG. 2 illustrates a communication network comprising a plurality of UAVs and a field-based sensor;

FIG. 3 is a flow diagram for a method performed by a remote server module for estimating the air quality in a geographical region comprising a field-based sensor fixed in the region;

FIG. 4 is a schematic diagram of a remote server module for estimating air quality in a geographical region; and

FIG. 5 is a schematic diagram of an unmanned aerial vehicle (UAV).

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

The following description is made for the purpose of illustrating the general principles of the present technology and is not meant to limit the inventive concepts claimed herein. As will be apparent to anyone of ordinary skill in the art, one or more or all of the particular features described herein in the context of one embodiment are also present in some other embodiment(s) and/or can be used in combination with other described features in various possible combinations and permutations in some other embodiment(s).

The measuring and forecasting of air quality is an important element of tackling air pollution such that high risk areas can be identified. Such measurements can also help determine trends in the types of areas that suffer from high risk air pollution levels. Typically, local authorities use distributed static air quality sensors to determine air pollution levels throughout a geographical region. For example, in urban environments, air quality sensors may be attached to lamp posts in predetermined locations. The air quality data received from a single air quality sensor may then be used to map the air quality of the region surrounding the air quality sensor.

An issue associated with using such a method is that the spatial resolution or granularity of air quality data associated with a region is limited by the density of static air quality sensors deployed in an area. Installing larger numbers of static quality sensors may be expensive, may require maintenance and calibration of an increased number of sensors and, in some instances, installing the required number of sensors may not be feasible. For example, in rural areas there may not be enough locations which are suitable to house a sensor.

Examples described herein achieve a finer granularity of air quality mapping around regions surrounding static field-based air quality sensors without the requirement for additional sensors to be installed. This is achieved by generating an air quality model for a region in which a fixed field-based air quality sensor is located. Using the air quality model, an estimation of air quality throughout the region radiating out from the field-based sensor can be made based on received air quality data from the field-based air quality sensor.

The air quality model is generated by deploying one or more mobile vehicles equipped with sensors in a geographical region surrounding a fixed field based sensor. In one example, the mobile vehicles are Unmanned Aerial Vehicles (UAVs). In an alternative example, the mobile vehicles are utility vehicles such as cars, vans, trucks or unmanned ground vehicles (UGVs). Air quality data is transmitted to a remote server module from both the field-based sensor and the mobile vehicle(s) which then generates or updates the air quality model using the received air quality data. The air quality is measured during the same time period by the mobile vehicle(s) and the field-based sensor. The model is generated based on the fact that when a particular air quality level was measured at the fixed field-based sensor, one or more particular air quality levels were measured at the same time by the one or more mobile vehicle(s) positioned at various locations in the region. Therefore, if the same air quality was measured at the field-based sensor at a different time, the air quality model would use the previous measurements performed by the mobile vehicles to provide an estimation as to what the air quality is in the region surrounding the field-based sensor.

Put differently, the remote server module uses the mobile vehicle's measurements to gather historical air quality data from the region surrounding a fixed field-based sensor, such that when a measurement is made at the fixed field-based sensor, the remote server module can make an estimation as to what the air quality is going to be in locations radiating out from the fixed field-based sensor using a model generated using the historical air quality data.

Therefore, instead of needing to install additional sensors in a geographical region, the remote server module instead uses the historical air quality data, which is built into an air quality model, to provide estimations of the air quality. In addition, the granularity of air quality forecasts generated by pre-existing networks of fixed field-based sensors can be increased using methods described herein. This is because the mobile vehicles can provide air quality data in locations between static field-based sensors which can be used to estimate air quality between the field-based sensors.

As an example scenario, the field-based sensor measures carbon monoxide CO to be at 1 ppm (part per million) at the north end of a residential street. There is not a field-based sensor at the south end of the residential street. To estimate the CO level at the south end of the street, the remote server module inputs the 1 ppm CO measurement into the air quality model. The air quality model at this stage may consult weather data at the present time. Because the air quality model was, at least partly, generated using data measured by a mobile vehicle which was positioned at the south end of the street, the air quality model knows how the air quality measurements typically compare between the north and south ends of the street. Thus, the air quality model can estimate the CO level at the south end of the street. For example, the CO level may be estimated to be 0.5 ppm at the south end, whereas previously it would have been estimated to be 1 ppm because the field-based sensor at the north end of the street was the nearest sensor.

In one example, the air quality model is also generated using climate data measured during the same time period as the air quality measurements were made by the field-based sensor and the mobile vehicle(s). This enables the generated air quality model to account for the influence of weather conditions on air quality measurements when estimating air quality. In some embodiments the weather data is measure by the mobile vehicle(s) and in other embodiment the weather data is retrieved from a different source (e.g. publicly accessible weather forecasts).

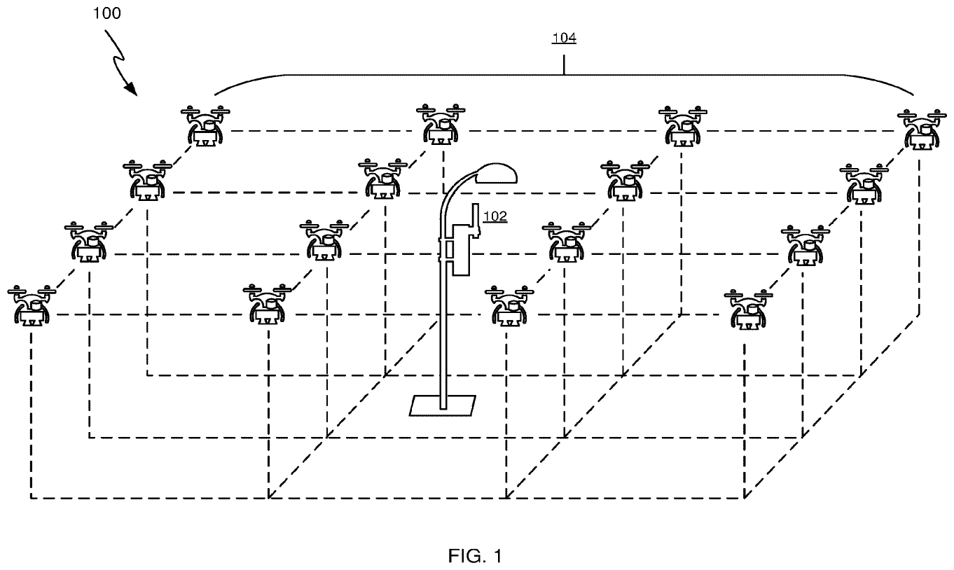

FIG. 1 of the accompanying drawings shows how one or more mobile vehicles (in the illustrated example of FIG. 1, the mobile vehicles are UAVs) are deployed in the region surrounding a fixed field-based sensor to build a network 100 of collaborating UAVs. A plurality of UAVs 104 are shown positioned at locations surrounding a fixed field based sensor 102. Note that FIG. 1 is an example only and is not intended to be limiting since fewer UAVs are used in other examples.

The UAVs 104 are equipped with sensors capable of providing a measurement of air quality in the location they are positioned. The air quality sensors are configured to detect at least one of: particulates (PM1.0, PM2.5, PM10), nitrogen dioxide (NO₂), nitric oxide (NO), sulphur dioxide (SO₂), hydrogen sulphide (H₂S), carbon dioxide (CO₂), carbon monoxide (CO), ozone (O₃), volatile organic compounds (VOCs), hydrocarbons, ammonia (NH₃) and additional air quality indicators not included in this non-exhaustive list. In another example the sensors also detect climate related variables. For example, the sensors also detect at least one of temperature, humidity, wind speed, wind gust speed, wind direction and pressure. In some cases the climate related variables include air flow caused by propellors of one or more UAVs.

In one example, the one or more UAV(s) 104 comprise means of moving the air quality sensors away from the propellors of the UAV(s) 104. For example, the sensors are lowered using a cord, arm or other retractable protection below the UAV By suspending one or more sensors from a UAV it is possible to mitigate the impact of downwards air flow from the UAV's propellors on air quality measurements.

In another example, the one or more UAV(s) 104 comprise sensors for measuring the direct air flow caused by the UAV's propellors. The direct air flow data is used to offset the influence of air flow on air quality measurements. In another example, the influence of air flow data on air quality measurements is also used in the generation of the air quality model. The influence of air flow data on air quality measurements may be determined empirically in a controlled environment such that air quality measurements are automatically correctable. That is, a UAV may be equipped with sensors to record the direct air flow caused by the UAV's propellers. This data may be used in calculations to offset the impact of downward air flow affecting/biassing the air quality measures. The impact of propeller airflow may be calculated in a lab environment and used as an input to the model, adjusting the air quality measure accordingly.

In another embodiment, the one or more UAV(s) 104 are positioned at predetermined locations relative to the fixed field-based sensor 102. For example, the UAVs are positioned relative to one another based on the range at which they can reliably transmit data to one another, however in another example the UAVs are positioned where they cannot communicate whilst performing measurements but store the air quality measurement data for transmission at another time. In another example, the one or more UAV(s) are positioned in areas of interest such as typically highly populated areas. The one or more UAV(s) 104 may be on a timetabled schedule for each position they take to measure air quality and may also position themselves in the vicinity of other field based sensors during a time period.

In another embodiment the UAV(s) 104 are positioned in a relay configuration whereby UAVs which are positioned at large distances transmit data via another UAV before the data reaches a remote server module. The relay configuration enables UAVs to cover a larger geographical area and perform measurements at the same time.

In another embodiment, the heights of the UAV(s) are varied at each position relative to the field-based sensor. A series of measurements taken at a plurality of heights such that the variation of the air quality compared to height can be included into the air quality model. Using the UAVs to measure the air quality at multiple heights means the height dependent trends can be calculated such that ground level air quality measurements can be extrapolated from measurements taken from above ground level. This is beneficial since UAVs collecting measurements at ground level may break local privacy laws as well as endangering members of the public.

In another embodiment, the one or more UAV(s) are performing tasks other than only measuring air quality (e.g., surveillance or package delivery) and perform air quality measurements whilst they perform said tasks. This has the effect of increasing the amount of air quality measurements to be used to generate the air quality model as an increased number of UAVs are utilised. In another embodiment, the one or more UAV(s) are positioned such that they avoid obstacles such as trees or buildings.

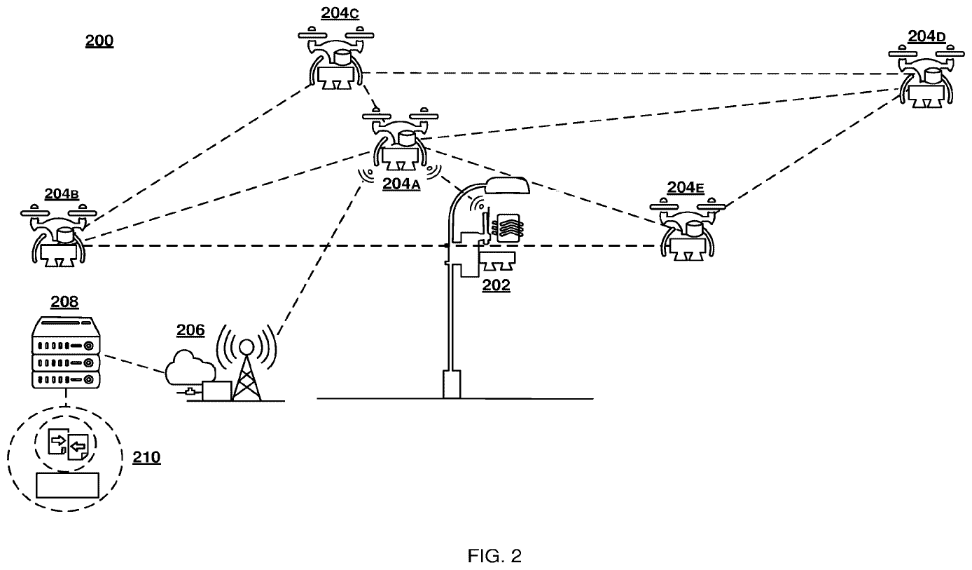

FIG. 2 of the accompanying drawings shows a communications network for generating an air quality model. A field-based sensor 202 is shown attached to a lamp surrounded by a plurality of mobile vehicles depicted as UAVs 204a-204e in FIG. 2. One UAV 204a is shown to be communicating with the field-based sensor 202, however additional UAVs 204b-204e may also communicate with the field-based sensor 202. The dotted lines connecting the UAVs 204a-204e indicate the UAVs in communication with one another. Note that it is not essential for the field-based sensor to be fixed to a lamp as it may be fixed in any suitable way to a location in a geographical region.

UAV 204a is shown to be connected to a network gateway 206 to which a remote server module 208 is connected where air quality data is collected and processed to generate air quality models 210 which are subsequently stored in the remote sever module 208. The remote sever module 208 serves as a centralised Internet of Things (IoT) data hub. In the example illustrated in FIG. 2, only 204a is shown to be connected to the network however any number of the UAVs 204a-204e may be connected.

In one example, UAV 204a is equipped with more sophisticated communications hardware compared to the other UAVs 204b-204e and/or the field-based sensor 202. The ‘heavy’ UAV 204a is thus capable of communicating with a network gateway from greater distances and serves as a relay point for the ‘light’ UAVs 204b-204e and the field-based sensor 202 to the network. Therefore, cheaper and more readily available UAVs (such as those whose primary purpose is not for air quality measurements) can be deployed as ‘light’ UAVs which are UAVs which are lighter in weight than the more sophisticated heavier weight UAVs. Additionally, only using one UAV to transmit the air quality data to the remote server module means that only one UAV is required to have authorised access to the remote server module. This improves efficiency at the remote server module 208 since only one data stream (that from the heavy UAV) associated with a geographical region needs to be verified and/or encrypted. This also improves security since the heavy UAV is more capable of performing more sophisticated encryption of the data sent to the remote server module.

The UAV(s) 204a-204e are equipped with a data store to store the data from the air quality and, in some examples, climate data. In one example, the UAV(s) 204a-204e store and send air quality and climate data at predetermined time intervals. To reduce memory requirements, the UAV(s) 204a-204e may discard data once it has been transmitted to the heavy UAV 204a or the remote server module 208. In addition, the UAVs may store air quality and climate data for a prolonged period and upload the data once the UAV is within transmittable range of a heavy UAV 204a or network gateway 206. This may be necessary when the UAVs are used to cover a large geographical area and are not always in transmittable range of other UAVs when performing measurements or when there are obstacles preventing data transmission.

The remote server module 208 receives the air quality data (and in some embodiments, climate data) from the one or more UAVs 204a-204e and, in some embodiments, the field-based sensor 202 via the communications network gateway 206. The remote server module 208 then stores the received data for processing such that an air quality model for a geographical region can be generated. The air quality model is capable of estimating the air quality in an area radiating out from a field-based sensor using a measurement from the field-based sensors. In one example, only a single measurement from the field-based sensor is requirement to make an estimation.

In some examples the model used to predict the air quality data is a trained machine learning model such as a neural network, random decision forest, support vector machine or other type of machine learning model. The machine learning model is trained in some examples using supervised learning as now explained.

Training data is collected through empirical measurement as a result of the historical data from the field-based sensor and the UAVs. The training data comprises thousands, tens of thousands or more of training examples. Each training example comprises:
measurements from the field-based sensor, a location of the field-based sensor, a time when the measurements were obtained, and measurements from one or more UAVs obtained at the same time as for the field-based sensor, and locations of the UAVs at the time of the measurements. The measurements from the field-based sensor comprise air quality data and optionally also climate data. The measurements from the UAVs comprise air quality data and optionally also climate data.

The training data pairs are used to train the machine learning model using any conventional supervised learning algorithm. In examples where the machine learning model is a neural network a supervised learning algorithm such as back propagation may be used. Parameters of the machine learning model are initialised to random values. One of the training examples is taken and the measurements from the field-based sensor are input to the neural network. A forward pass through the neural network is computed to produce an output from an output layer of the neural network. The output is compared with the measurement values from the UAV(s) using a loss function. Any suitable loss function is used such as a squared error loss or other loss function. The loss function is used to update the values of the parameters of the neural network during a backward pass of the neural network. The values of the parameters are updated so as to reduce the loss computed from the loss function. Another of the training examples is taken and the process of computing a forward pass, loss function and backward pass to update the parameter values is done. This is repeated for more of the training examples until convergence is reached. Convergence is where the values of the parameters do not change or change by less than a threshold amount; or where a specified number of training examples have been processed.

In some examples, the measurements are mapped to a multi-dimensional space, in which measurements which are similar are close together in the space, before being input to the neural network. In this way, one or more measurements to be input to the neural network for a particular time instant are converted into vector format where the vector is an embedding vector of the multi-dimensional space. By using embedding vectors it is possible to input air quality and climate measurements into the neural network despite these values being in different units and formats and being of different quantities.

In some examples the model used to predict the air quality data is a rule based system. Rules in the rule based system are used to select historical data and to use one or more of: regression, extrapolation, interpolation, inference to predict air quality data for a particular time and location in the geographical region. In an example, the rule based model is queried with a specified location in the geographical region relative to the field-based sensor. Rules are used to select historical data such as by searching for historical data obtained from UAVs close to the specified location, or by selecting historical data obtained from UAVs at a time where the measurements from the field-based sensor were similar to those currently obtained from the field-based sensor. Suppose relevant historical data is obtained from two UAVs either side of the specified location. Interpolation may be used to interpolate between the historical data from the two UAVs and the current data from the field-based sensor in order to predict air quality data at the specified location.

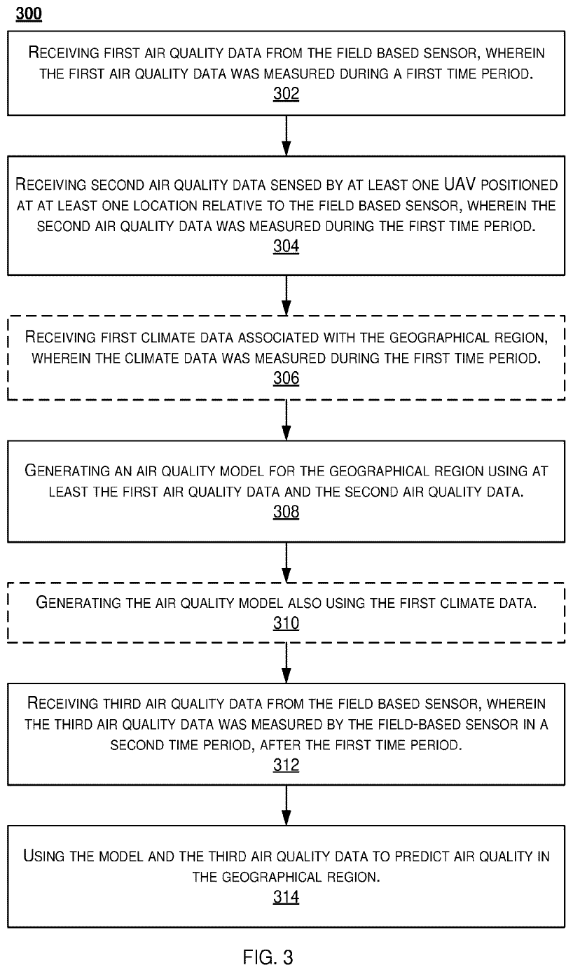

FIG. 3 of the accompanying drawings shows a flow diagram for a method 300 performed by a remote server module for estimating the air quality in a geographical region comprising a field-based sensor fixed in the region. UAVs are used as the mobile vehicles in the method 300 although other mobile vehicles may be used. At operation 302 of the method 300 the remote server module receives first air quality data from the field-based sensor, wherein the first air quality data was measured during a first time period. The first time period may be a day or an hour for example, where the air quality in an urban area is being monitored. In other situations, where air quality in an indoor environment such as a manufacturing facility is being monitored the time period may be a minute or fraction of an hour. Air quality measurements may be collected every minute or at any other time interval during the first time period. The length of the first time period is configurable depending on the application to which the method 300 is applied.

At operation 304 of the method 300 the remote server module receives second air quality data sensed by at least one UAV positioned at at least one location relative to the field-based sensor, wherein the second air quality data was measured during the first time period.

In some embodiments, at operation 306 of the method 300 the remote server module receives first climate data associated with the geographical region, wherein the climate data was measured during the first time period. The first climate data is received from any entity such as a climate data service in the cloud or any other source.

At operation 308 of method 300 the remote server module generates an air quality model for the geographical region using at least the first air quality data and the second air quality data. In an example, the air quality model is generated by storing the first air quality data in association with the second air quality data in a store of historical data. The historical data is then used together with rules to form the model, where the model is a rule-based system. In another example, the model is a trained machine learning model and the first air quality data and the second air quality data are used as a training example during supervised training of the model. In this case, the model may be trained using many more training data examples collected empirically in the same way.

In some examples, at operation 310 of the method 300 the remote server module generates the air quality model also using the first climate data.

At operation 312 of the method 300 the remote server module receives third air quality data from the field-based sensor, wherein the third air quality data was measured in a second time period, after the first time period. The second time period may be a second or minute or any other duration such as a few days. Air quality measurements may be predicted every minute or at any other time interval during the second time period. In some cases the air quality predictions are made continuously as data from the field-based sensor is received, which may be at the frequency the air quality is measures, or may be according to batches of air quality measurements batched up and sent by the field-based sensor at 5, 10 or other sized time intervals. The second time period may be immediately consecutive to the first time period. In other examples, the second time period is separated by a gap from the first time period. The first and second time periods are independent of one another.

At operation 314 of the method 300 the remote server module uses the model and the third air quality data to predict air quality in the geographical region. In an example, the third air quality data and a location in the geographical region is used to query a trained machine learning model which returns a prediction of the air quality at the location. The model may be repeatedly queried using the third air quality data and different locations in the geographical region in order to build up a map of air quality over the geographical region. In another example, the third air quality data and a location in the geographical region is used to query a rule-based model of the air quality data. In this case, historical data in the rule based model is searched to find historical air quality data at the field-based sensor which is similar to the third air quality data. The historical air quality data which is found is stored in association with air quality data at other locations in the geographical region and that air quality data is returned in response to the query.

In various examples, climate data is also measured in association with the third air quality data. In operation 314 the remote server module queries the model using the third air quality data and the climate data and a specified location. Where the model is a trained machine learning model the third air quality data and the climate data are input to the trained machine learning model together with the specified location. The trained machine learning model predicts air quality data at the specified location. This may be repeated for other locations in order to build up a map of predicted air quality over the geographical region. Where the model is a rule-based model the third air quality data is used to search for historical air quality data as explained above. The climate data is taken into account by rules which are used to compute the predicted air quality data.

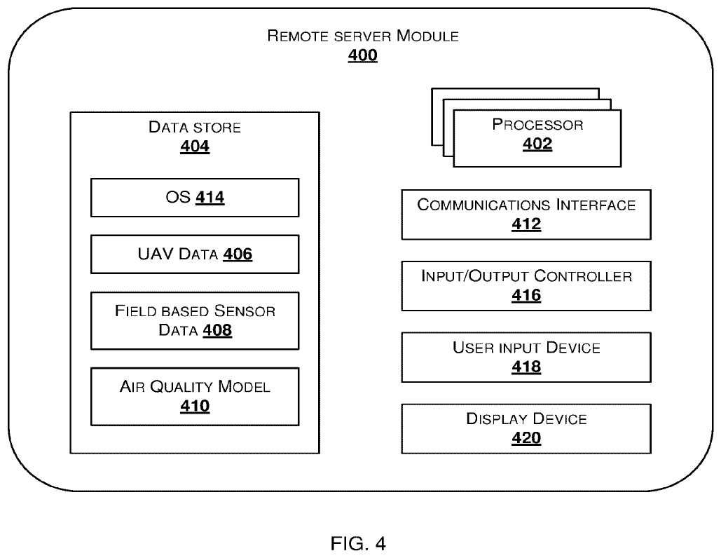

FIG. 4 of the accompanying drawings shows a computing device capable as serving as a remote server module 400. The remote server module 400 comprises one or more processors 402 which are microprocessors, controllers or any other suitable type of processors for processing computer executable instructions to control the operation of the device in order to perform the methods of FIG. 3. In some examples, for example where a system on a chip architecture is used, the processors 402 include one or more fixed function blocks (also referred to as accelerators) which implement a part of the method of FIG. 3 in hardware (rather than software or firmware). That is, the methods described herein are implemented in any one or more of software, firmware or hardware. The remote server module 400 has a data store 404 holding UAV air quality data 406, field-based sensor data 408 and the air quality model 410. The remote server module 400 has a communications interface 412 for communicating with UAVs and field-based sensors. Platform software comprising an operating system 414 or any other suitable platform software is provided at the computing-based device to enable application software to be executed on the device. Although the computer storage media (data store 404) is shown within the remote server module 400 it will be appreciated that the storage is, in some examples, distributed or located remotely and accessed via a network or other communication link (e.g. using communication interface 412).

In some embodiments, the remote server module 400 also comprises an input/output controller 416 arranged to output display information to a display device 420 which may be separate from or integral to the remote server module 400. The display information may provide a graphical user interface. The input/output controller 416 is also arranged to receive and process input from one or more devices, such as a user input device 418 (e.g. a mouse, keyboard, camera, microphone or other sensor). In some examples the user input device 418 detects voice input, user gestures or other user actions. In an embodiment the display device 420 also acts as the user input device 418 if it is a touch sensitive display device. The input/output controller 418 outputs data to devices other than the display device in some examples.

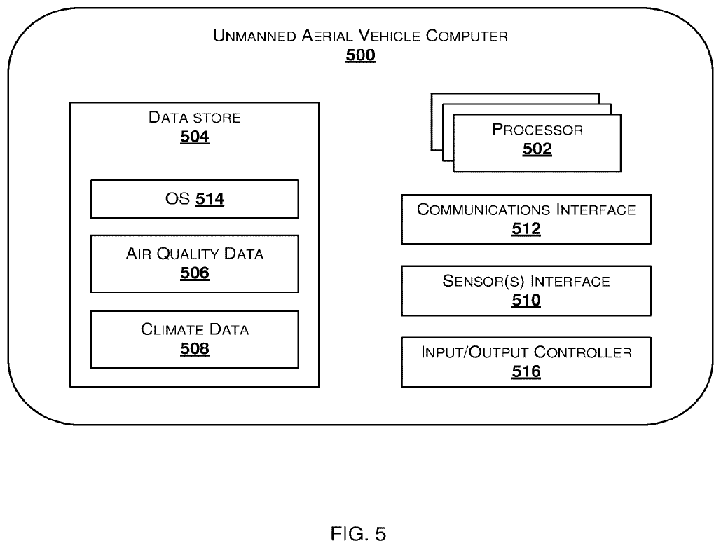

FIG. 5 of the accompanying drawings shows a computing device housed within a UAV 500 such that the tasks assigned to the UAV in FIG. 3 can be performed.

The UAV computer 500 comprises one or more processors 502 which are microprocessors, controllers or any other suitable type of processors for processing computer executable instructions to control the operation of the device in order to perform the methods of FIG. 3. In some examples, for example where a system on a chip architecture is used, the processors 502 include one or more fixed function blocks (also referred to as accelerators) which implement a part of the method of FIG. 3 in hardware (rather than software or firmware). That is, the methods described herein are implemented in any one or more of software, firmware or hardware. The UAV computer 500 has a data store 504 holding air quality data 506 and climate data 508. The UAV computer 500 has a communications interface 512 for communicating with other UAVs, field-based sensors and the remote server module 400. Platform software comprising an operating system 514 or any other suitable platform software is provided at the computing-based device to enable application software to be executed on the device. Although the computer storage media (data store 504) is shown within the UAV computer 500 it will be appreciated that the storage is, in some examples, distributed or located remotely and accessed via a network or other communication link (e.g. using communication interface 512). The UAV computer 500 comprises a sensor(s) interface 510 configured to control the sensors configured to measure air quality and/or climate conditions. In some embodiments, the UAV computer 500 also comprises an input/output controller 516 arranged to receive and process inputs from the one or more sensors installed into the UAV. The input/output controller 516 is also arranged to receive and process user input.

Any reference to ‘an’ item refers to one or more of those items. The term ‘comprising’ is used herein to mean including the method blocks or elements identified, but that such blocks or elements do not comprise an exclusive list and an apparatus may contain additional blocks or elements and a method may contain additional operations or elements. Furthermore, the blocks, elements and operations are themselves not impliedly closed.

The steps of the methods described herein may be carried out in any suitable order, or simultaneously where appropriate. The arrows between boxes in the figures show one example sequence of method steps but are not intended to exclude other sequences or the performance of multiple steps in parallel. Additionally, individual blocks may be deleted from any of the methods without departing from the spirit and scope of the subject matter described herein. Aspects of any of the examples described above may be combined with aspects of any of the other examples described to form further examples without losing the effect sought. Where elements of the figures are shown connected by arrows, it will be appreciated that these arrows show just one example flow of communications (including data and control messages) between elements. The flow between elements may be in either direction or in both directions.

Where the description has explicitly disclosed in isolation some individual features, any apparent combination of two or more such features is considered also to be disclosed, to the extent that such features or combinations are apparent and capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the invention.

1. A method, performed by a remote server module, for estimating the air quality in a geographical region comprising a field-based sensor fixed in the region, the method comprising:
receiving first air quality data from the field-based sensor, wherein the first air quality data was measured during a first time period;
receiving second air quality data sensed by at least one mobile vehicle positioned at at least one location relative to the field-based sensor, wherein the second air quality data was measured during the first time period;
generating an air quality model for the geographical region using at least the first air quality data and the second air quality data;
receiving third air quality data from the field-based sensor, wherein the third air quality data was measured by the field-based sensor in a second time period, after the first time period; and
using the model and the third air quality data to predict air quality in the geographical region.

2. The method as claimed in claim 1, further comprising:
receiving first climate data associated with the geographical region, wherein the climate data was measured during the first time period; and wherein
generating the air quality model further comprises using the first climate data.

3. The method as claimed in claim 1, further comprising:
wherein the third air quality data was measured at a time when air quality data from mobile vehicles in the geographical region was unavailable.

4. The method as claimed in claim 3, further comprising:
receiving second climate data associated with the geographical region, wherein the second climate data was measured during the second time period; and
using the second climate data, the model and the third air quality data to predict air quality in the geographical region.

5. The method as claimed in claim 1, wherein the first and/or second climate data is measured by the at least one mobile vehicle.

6. The method as claimed in claim 1, wherein the mobile vehicle is an Unmanned Aerial Vehicle, UAV, and wherein the second air quality data is measured at a plurality of heights at the at least one location of the at least one UAV.

7. The method as claimed in claim 6, further comprising:
calculating a height dependence of air quality in the geographical region using the second air quality data measured at the plurality of heights; wherein
generating the air quality model further comprises using the height dependence of air quality.

8. The method as claimed in claim 1, wherein one or more lighter weight mobile vehicles of the at least one mobile vehicles and/or the field-based sensor are configured to send air quality data to a heavier weight mobile vehicle, wherein the first, second and third air quality data is received from the heavier weight mobile vehicle.

9. The method as claimed in claim 1, wherein the air quality model is a machine learning model.

10. The method as claimed in claim 1 comprising receiving a measurement of air flow caused by propellors of the mobile vehicle and wherein predicting the air quality comprises taking into account the air flow causes by the propellors.

11. A remote server module for estimating the air quality in a geographical region comprising a field-based sensor fixed in the region, the remote serve module configured to:
receive first air quality data from the field-based sensor, wherein the first air quality data was measured during a first time period;
receive second air quality data sensed by at least one mobile vehicle positioned at at least one location relative to the field-based sensor, wherein the second air quality data was measured during the first time period;
generate an air quality model for the geographical region using at least the first air quality data and the second air quality data;
receiving third air quality data from the field-based sensor, wherein the third air quality data was measured by the field-based sensor in a second time period, after the first time period; and
using the model and the third air quality data to predict air quality in the geographical region.

12. The remote server module as claimed in claim 11, further configured to:
receive first climate data associated with the geographical region, wherein the climate data was measured during the first time period; and wherein
generating the air quality model further comprises using the first climate data.

13. The remote server module as claimed in claim 10, wherein the second air quality data was measured by a plurality of mobile vehicles in the geographical region and wherein the predicting air quality in the geographical region comprises predicting air quality at locations in the geographical region disparate from the field-based sensor and the mobile vehicles.

14. The remote server module as claimed in claim 11, wherein the second air quality data was measured by suspending a sensor from a UAV.

15. A computer implemented method for estimating air quality at a specified time and location in a geographical region comprising a field-based sensor fixed in the region, the method comprising:
receiving air quality data from the field-based sensor, wherein the air quality data was measured by the field-based sensor at the specified time;
inputting the air quality data and the location to a trained model to predict the air quality at the specified time and location;
wherein the location is different from the location of the field-based sensor and wherein the model has been trained using air quality data measured by the field-based sensor and at least one mobile vehicle in the region.
